(19) United States
(12) Patent Application Publication
(10) Pub. No.: US 2008/0082028 A1
(43) Pub. Date: Apr. 3, 2008

(54) Massaging Apparatus

(76) Inventor: James Blevins, Eagle Point, OR (US)

Correspondence Address: Jay M. Schloff;Intellipex PLLC, Suite 245, 30200 Telegraph Road, Bingham Farms, MI, 48025, US

(21) Appl. No.: 11/935,436

(22) Filed: Nov. 6, 2007

Related U.S. Application Data
(63) Continuation-in-part of application No. 11/540,774, filed on Sep. 29, 2006, now abandoned.

(51) Int. Cl. A61H 1/00; A61F 5/00

(52) U.S. Cl. 601/72; 600/38

(57) ABSTRACT

Disclosed is a massaging apparatus which incorporates motion, friction, and heat. The massaging apparatus comprises a vagina, a vibrator coupled to the vagina and a pair of hand grips disposed on both sides of the vibrator. The vagina is in the form of an elongated hollow sheath having a first end and a second end opposite to each other. The first end is open and the second end is closed. The vibrator is at least one of a hand held electric vibrator and a hand held battery powered vibrator. The massaging apparatus may be used by a single male, a single female, simultaneously by a male and female, and simultaneously by two females.

### RELATED APPLICATIONS

This application is a continuation-in part of, and is entitled to the benefit of the earlier filing date and priority of, U.S. patent application Ser. No. 11/540,774 filed on Sep. 27, 2006, which names the same inventor as the current application, the disclosure of which is hereby incorporated by reference.

### FIELD OF THE INVENTION

The present invention relates to the field of sexual devices used by males and females, either alone or together, for the purpose of erotic stimulation and climax. In particular, the present invention relates to a massaging apparatus with electrical percussive vibrators.

### BACKGROUND OF THE INVENTION

The desire for sex and orgasm is a primal need among humans, indeed among all living creatures. Many times, for whatever reason, a person finds themselves without a suitable partner. Sometimes even with a partner, it is desirable to experiment and try something new and different. Enter the need for a portable, light weight, hygienic, gender neutral, safe, reliable, and moderately priced sexual device.

With the advent of dry cell batteries and various plastics, there has been an explosion of sex devices and toys. But even some of the heavily researched and engineered devices end up being cumbersome and unsatisfying in what they accomplish.

Generally, sex toys, including vibrators, available in the market are hand held vibrators, and most of them rely on small quickly depleted dry cell batteries. Additionally, most of such vibrators are for women.

For example, U.S. Patent Publication No. 2005/0228219 discloses a device designed to take rotary power from an external power source such as a cordless electric screwdriver and transmit that energy into both rotary and reciprocating linear activation of sexual devices used by both males and females for the purpose of masturbation and auto-stimulation.

U.S. Patent Publication No. 2005/0113636 discloses a stimulation device having a reciprocating projecting member that projects out of a slot through the housing. A motor in the housing drives a linkage which converts the motor's rotation into reciprocation. The linkage reciprocates the projecting member. The housing is relatively large and mounts on a horizontal surface. Its weight maintains it in one place during use.

U.S. Patent Publication No. 2005/0049453 discloses “three ways to achieve vibrations or other rhythmic motions within a sexual stimulator via movement of hydraulic fluid into and out of said sexual stimulator.” However, the three ways described in the publication involve additional equipment and cost.

U.S. Patent Publication No. 2004/0176710 discloses a massage device for creating a massaging action. The massage device may include at least two massaging projections mounted in relation to a flexible fluid supply conduit, such as a shower hose. The device may include a chamber for containing an impeller, and the fluid supply conduit may include an outlet for fluid to contact the impeller and cause the impeller to rotate. At least one weight is disposed in the head and is coupled to the impeller eccentric to a rotational axis of the impeller for moving the projection in an oscillatory motion, which creates the massaging action.

U.S. Pat. No. 6,926,666 discloses a penile tensioning system, device, kit, and methods of using same. The penile tensioning device includes a constriction body region of bendable and stretchable material having outer surface peripheries and inner surface peripheries substantially surrounding a medial aperture. The inner surface peripheries are adapted to substantially surround and abuttingly contact outer surface peripheries of a proximal portion of a penis for substantially evenly applying constriction pressure around the circumference of the penis when positioned thereon. The system includes the device and a vacuum device to use in the positioning of the device.

U.S. Pat. No. 6,890,293 discloses a driving apparatus of a massaging device for sexual organs comprising a pedestal, a driving device fixed detachably on the pedestal, and moving means provided on the driving device, to which a massaging device for sexual organs is attached. The pedestal of planar plate on which a hip of the user is set. The driving device further comprises a motor, a rotary cam, and gear trains for converting the rotary motion of the motor.

U.S. Pat. No. 5,458,559 discloses an artificial vagina including a sheath made from a flexible waterproof material. The sheath tapers from one end to the other, the wider end having a resealable opening closed by a screw-threaded stopper. Projecting from the inner end of stopper is an eye loop connected to a hook which swivels on wire loop secured to tab. The tapered end of sheath has a soft flexible undulating wall and a bulbous portion from which extends an annular membrane, the peripheral edge of which has a reinforced elastic rib. When assembled with the hook connected to wire loop a fluid chamber is formed and filled with warm water forming a chamber. Aperture is formed in wall to connect with bulbous portion for the passage of the water. The design of the device facilitates critical and necessary cleansing for reuse.

U.S. Pat. No. 5,421,808 discloses a male organ conditioning appliance which is a tubular self-contained, battery-operated external vacuum device which includes an electric motor with an eccentric output shaft coupled with a reciprocating diaphragm pump in a common housing with the electric motor and batteries. Vacuum devices, it turns out, are quite popular in the prior art.

U.S. Pat. No. 5,397,294 discloses a health aid device that is worn around the erect penis, is held in place by a belt worn around the man's waist. The device generates a high frequency vibration so as to shorten the excitement period of the female and the male and female can thus obtain the same level of satisfaction during sexual intercourse.

### SUMMARY OF THE INVENTION

The general purpose of the present invention is to provide a massaging apparatus configured to include all the advantages of the prior art, and to overcome the drawbacks of the prior art. The massaging apparatus proposed by the present invention, is characterized by a particularly apposite effect on the male penis and by a fairly simple form of construction.

In one aspect, the present invention provides a massaging apparatus. The massaging apparatus comprises a vagina, a vibrator coupled to the vagina and a pair of hand grips disposed on both sides of the vibrator. The vagina is in the form of an elongated hollow sheath having a first end and a second end opposite to each other. The first end is open and the second end is closed. The vibrator is at least one of a hand held electric vibrator and a hand held battery powered vibrator. The vagina further comprises a vent hole configured at a tip of the second end to remove trapped air formed within the vagina upon inserting a penis into the vagina. Further, the vagina may be made of a transparent or an opaque material. The massaging apparatus is capable of being used by a single male.

In an additional aspect, the massaging apparatus further comprises two dildos removably attached to the first end and the second end of the vagina such that the massaging apparatus is capable of being used by two females simultaneously.

In another aspect, the present invention provides a massaging apparatus capable of being used by a single female. The massaging apparatus comprises a vagina, a vibrator coupled to the vagina, a pair of hand grips disposed on both sides of the vibrator and a dildo. The vagina in the form of an elongated hollow sheath having a first end and a second end opposite to each other, the first end is open and the second end is closed. The vibrator is at least one of a hand held electric vibrator and a hand held battery powered vibrator. The dildo is removably attached to the first end of the vagina.

In yet another aspect, the present invention provides a massaging apparatus capable of being used by a single male and a single female simultaneously. The massaging apparatus comprises a vagina, a vibrator coupled to the vagina, a pair of hand grips disposed on both sides of the vibrator and a dildo. The vagina in the form of an elongated hollow sheath having a first end and a second end opposite to each other. The first end is open and the second end is closed. The vibrator is at least one of a hand held electric vibrator and a hand held battery powered vibrator. The dildo is removably attached to the second end of the vagina.

These together with other aspects of the present invention, along with the various features of novelty that characterize the invention, are pointed out with particularity in the claims annexed hereto and form a part of this invention. For a better understanding of the invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The attached drawings disclose an illustrated embodiment of the invention and form a part of the specification. It is to be noted, however, that the drawings illustrate only certain embodiments of the invention and are therefore not to be considered limiting of the invention in scope as it may include other effective embodiments as well.

FIG. 1 is a perspective view of a massaging apparatus including a vagina, a vibrator body, an adjustable speed control switch and a heat control switch, according to an embodiment of the present invention;

FIG. 2 is a bottom view of the vagina showing mounting cups attached to the vagina, according to an embodiment of the present invention;

FIG. 3 is a cross-sectional view taken along line 3-3 of FIG. 2;

FIG. 4 is a cross-sectional view taken along line 4-4 of FIG. 2;

FIG. 5 is a side elevation view of a dildo-1, according to an embodiment of the present invention;

FIG. 6 is a front elevation view of a dildo-1 flange, according to an embodiment of the present invention;

FIG. 7 is a front elevation view of an insert flange, according to an embodiment of the present invention;

FIG. 8 is a side elevation view showing the dildo-1 coupled to a first end of the vagina, according to an embodiment of the present invention;

FIG. 9 is a side elevation view showing a dildo-2 coupled to a second end of the vagina, according to an embodiment of the present invention; and

FIG. 10 is a side elevation view showing the dildo-1 coupled to the first end of the vagina and the dildo-2 coupled to the second end of the vagina, according to an embodiment of the present invention.

Like reference numerals refer to like parts throughout the description of several views of the drawings.

### DETAILED DESCRIPTION OF THE INVENTION

The exemplary embodiments described herein detail for illustrative purposes are subject to many variations in structure and design. It should be emphasized, however, that the present invention is not limited to a particular massaging apparatus, as shown and described. It is understood that various omissions and substitutions of equivalents are contemplated as circumstances may suggest or render expedient, but these are intended to cover the application or implementation without departing from the spirit or scope of the claims of the present invention. Also, it is to be understood that the phraseology and terminology used herein is for the purpose of description and should not be regarded as limiting.

The present invention relates to various lubricious aspects, both tactile and visual, of human sex play and masturbation. Embodiments have been made for use by a single male, a single female, simultaneously by a male and female, and simultaneously by two females.

The present invention provides a massaging apparatus capable of providing a massage that incorporates motion, friction, and heat in an attractive, portable, and economical package. The combination of totally controllable massage and a sustainable controlled heat source has, until now, eluded the sex aid industry. The massaging apparatus of the present invention is especially quiet at the lower speeds at which it may be operated.

More specifically, the present invention provides a massaging apparatus comprising a vagina, a plurality of dildos and a vibrator. The vagina is in the form of an elongated hollow cylindrical sheath having a first end and a second end opposite to the first end. The first end is open and the second end is closed. The massaging apparatus is further designed such that the bottom of the vagina is connected to an electric power unit (herein referred to as a ‘vibrator’). The vibrator is having means for exciting two parallel massage nodes attached to the vagina and produce percussive motion in an asynchronous manner.

Further objects, advantages and features of the present invention will be understood from a discussion of the drawings and the claims.

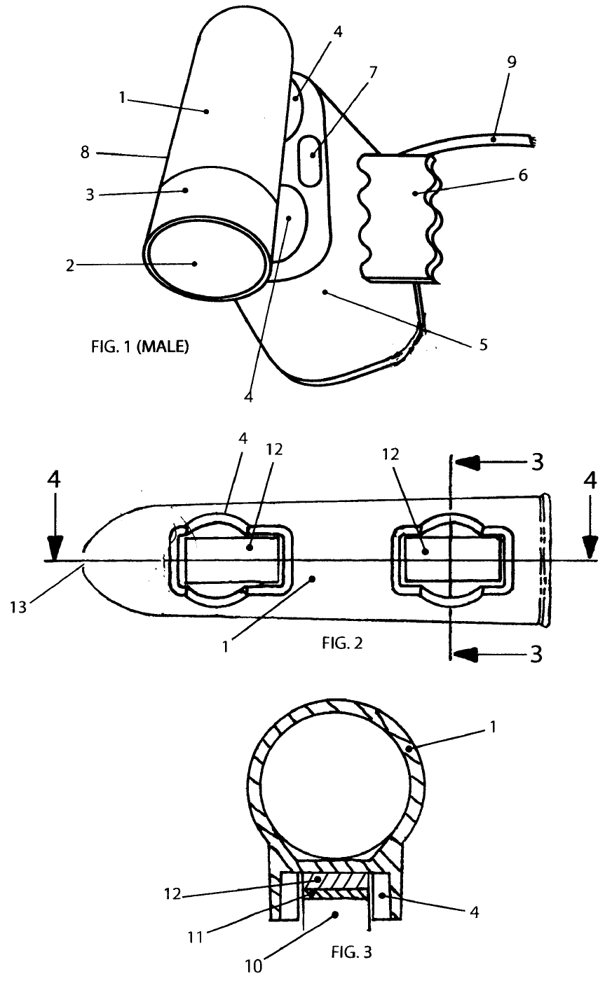

Referring to FIG. 1, a vagina 1 and a vibrator 5 of a massaging apparatus is shown. The vagina 1 has a first end 2. The first end 2 is an opening which enables in inserting a user's penis into the vagina 1. The vagina 1 may be made from clear polycarbonate plastic to heighten the visual appeal. Alternatively, various electomeric plastics may be used as a substitute for the polycarbonate. The variations in size, texture, and color may be derived either by direct substitution for the vagina 1 or by using insertable sleeves. The composition of the vagina 1 results in a massaging apparatus which is durable, impervious to various lubricants, and easy to clean. Due to the configuration of the massaging apparatus, the vagina 1 vibrates asynchronously with the vibrator 5, and the vagina 1 may be used by a single male for sexual stimulation. In one embodiment of the present invention, the vagina 1 is made of an opaque material.

Additionally, at a proximal end of the vagina 1 a piece of closed-cell foam 3 is located. The closed-cell foam 3 is attached to the upper ⅓ of the sheath. The closed-cell foam 3 closely simulates the sensation of the Kegel muscle in a woman's vagina. Mounting cups 4 are molded onto the vagina 1 and enables in attaching the vagina 1 to the vibrator 5. The massaging apparatus further comprise a pair of hand grips 6 disposed on both sides of the vibrator 5. More specifically, the hand grips 6 are molded onto both sides of the vibrator 5 for ease of manipulation. A sliding on-off-speed switch 7 of the vibrator 5 gives the user ability to achieve the speed desired from 0-2000 pulses per-minute, which is from a slow “wobble” to an uncomfortable “buzz”. On the one side of the vibrator 5, a heat switch 8 is configured. In one embodiment of the present invention, the vibrator 5 is an AC powered vibrator and a supply cord 9 is shown connected to the lower-right side of the vibrator 5.

FIG. 2 is a bottom view of the vagina 1, showing the particular circular-rectangular configuration of the mounting cups 4. The circular region of the cups 4 attach to the vibrator nodes 10 and the rectangular region of the cups 4 receive heat strips 12. The heat strips 12 are metal heat strips that derive heat from heat conductors 11 and transfer the heat through the bottom of the vagina 1 and thence to the user.

FIG. 3 is a cross-sectional view of the vagina 1 taken along section line 3-3 of FIG. 2, showing the integral mounting cups 4 which are attached to the circular vibrator nodes 10. Further, shown are the heat conductors 11, which are fastened to the top of the vibrator nodes 10 and the heat strips 12. The heat strips 12 rest directly on the heat conductors 11 and the said heat conductors 11 being activated by sliding the heat switch 8. The type and thickness of the metal for the heat strips 12 is carefully chosen so as to provide the user with a pleasant experience. In one embodiment, the metal is ⅛″ thick aluminum.

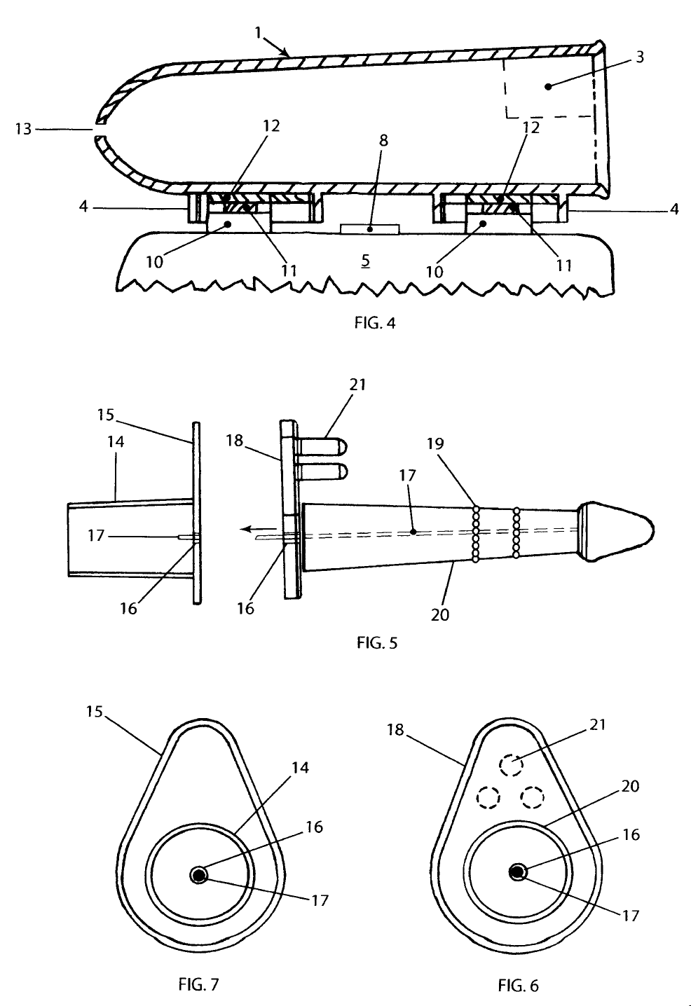

Referring to FIG. 4, a cross-sectional elevation view of the vagina 1 taken along line 4-4 of FIG. 2 is depicted. In the FIG. 4, shown are the vibrator 5, the vibrator nodes 10, the heat conductors 11 and the heat strips 12.

Another aspect of the present invention is shown by the FIG. 4, the taper designed vagina 1. The taper designed vagina 1 leads the erect male penis to wedge against the inside of the vagina 1 once it is past the half-way mark, thereby creating a partial vacuum therewithin. The partial vacuum results in a noticeable tugging at the glands penis, as either the vagina 1 is moved longitudinally or the user thrusts his pelvis. A vent hole 13 is configured at a tip of the closed second end for removing trapped air formed within the vagina 1 upon inserting a penis into the vagina 1. The dimensions of the vagina 1 may be of any size. In one embodiment, the dimensions of the vagina 1 are configured to receive the erect penis of an average-sized adult male.

Beginning with FIG. 5, another embodiment of the invention is shown. In this embodiment, a massaging apparatus that can be shared by a male and a female or by two females is disclosed. There are various ways to attach the female adaptors to the vagina 1, for example threads, twist locks, snap locks, bayonet fittings, tapered plug, and meshing grooves/splines. The latter two methods, tapered plug and meshing grooves/splines, are illustrated in FIGS. 5, 8, and 9. Further, in FIG. 5 a female insert 14 is shown. The female insert 14 has a snug, tapered fit and is designed to insert into the first end 2 of the vagina 1 up to an insert flange 15. The insert flange 15 is designed such that the insert flange 15 mates to a dildo-1 flange 18 and gets cemented with the dildo-1 flange 18. The female insert 14 and the insert flange 15 are molded monolithically. Similarly, a dildo-1 20 and the dildo-1 flange 18 are molded monolithically.

The dildo-1 20 and dildo-2 28 may be made of any material providing the feel of an artificial penis to a user. Suitable materials include different polymeric materials, plastic-jelly materials, simulated skin materials, rubber, silicone, and the like. In one embodiment, the dildo-1 20 is made of soft silicone, and the dildo-1 20 further comprises a method for stiffening the dildo-1. The method for stiffening the dildo-1 20 comprises a stiffening rod 17 of the correct stiffness, which is inserted through a rod insert opening 16 formed into the insert flange 15 and dildo-1 flange 18. Once the insert flange 15 and dildo-1 flange 18 are joined according to an arrow shown in the FIG. 5, the motion of the vibrator nodes 10 is transferred to the dildo-1 20. Further, a dildo-2 28 is configured in similar manner to the dildo-1 20 for stiffening.

The dildo-1 20 and dildo-2 28 have several elements that contribute to the female's arousal and sexual satisfaction. The dildo-1 20 and dildo-2 28 are molded with two rings of exciter knobs 19. Additionally, a group of three soft clitoral ticklers 21 are molded onto the dildo-1 flange 18 and a dildo-2 flange 27. The flanges 18, 27 and the dildos 20, 28 have different hardness, thus combination piece is molded using the “overmold” technique, such that a part of an object is molded of one plastic and then transferred mechanically to another mold where a second type of plastic is used to complete the desired object.

FIG. 6 and FIG. 7 are front elevation views of the insert flange 15 and the dildo-1 flange 18, respectively, showing the stiffening rod 17 and the clitoral ticklers 21.

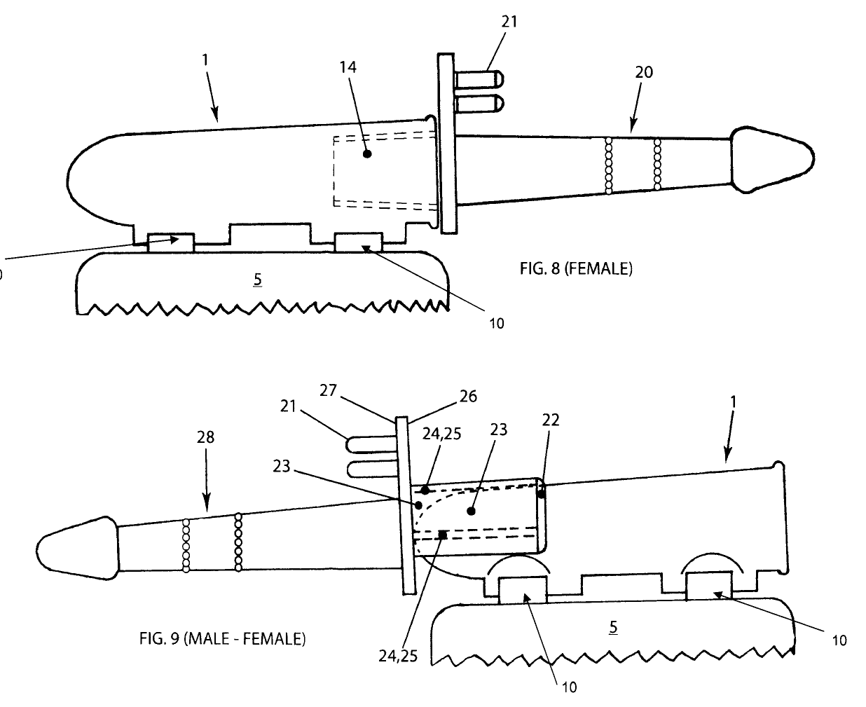

Referring to FIG. 8, in another embodiment of the present invention, a massaging apparatus adapted for use by a single female is shown. In the present embodiment, the female insert 14 is firmly pressed into the vagina 1, such that the dildo-1 20 and the vagina 1 behave as a single unit. Upon selecting the speed for the vibrator nodes 10, a vibration corresponding to the selected speed is transmitted to the entire female attachment. Due to the configuration of the massaging apparatus, the vagina 1 and the dildo-1 20 vibrate asynchronously with the vibrator 5, and the vagina 1 and the dildo-1 20 may be used by a single female for sexual stimulation.

FIG. 9 illustrates another embodiment of the present invention, relating to a massaging apparatus which can be used by a man and a woman at the same time, such that both the man and the woman receive the stimulus of each other's company as well as the varied stimulus of the massaging apparatus. In this embodiment, the closed second end of the vagina 1 is molded with a thickened sidewall that extends partially over the body of the vibrator node 10, near to the second end of the vagina 1. Three grooves 24 are molded into the externally thickened sidewall portion at equal distances around the upper two-thirds of the vagina 1. A tapered dildo-2 sleeve 23 of the dildo-2 28 is molded with three matching splines 25, such that the splines 25 are molded at corresponding distance around the inside perimeter of the dildo-2 sleeve 23. Due to the configuration of the massaging apparatus, the vagina 1 and the dildo-2 28 vibrate asynchronously with the vibrator 5, and the vagina 1 and the dildo-1 28 may be used simultaneously by a male and a female for sexual stimulation.

The dildo-2 28 is molded monolithically with the dildo-2 flange 27. The dildo-2 flange 27 is mated and cemented to a sleeve-flange 26. The dildo-2 sleeve 23 is slid over the second end of the vagina 1 and the splines 25 engages the grooves 24, such that the dildo-2 28 is locked onto the vagina 1 and the motion of the vibrator nodes 10 is transferred to the vagina 1 and the dildo-2 28. The sleeve-flanges 26 and dildo-2 flange 27 are identical in shape and function, although slightly larger, than insert flange 15 and dildo-1 flange 18 in FIG. 5.

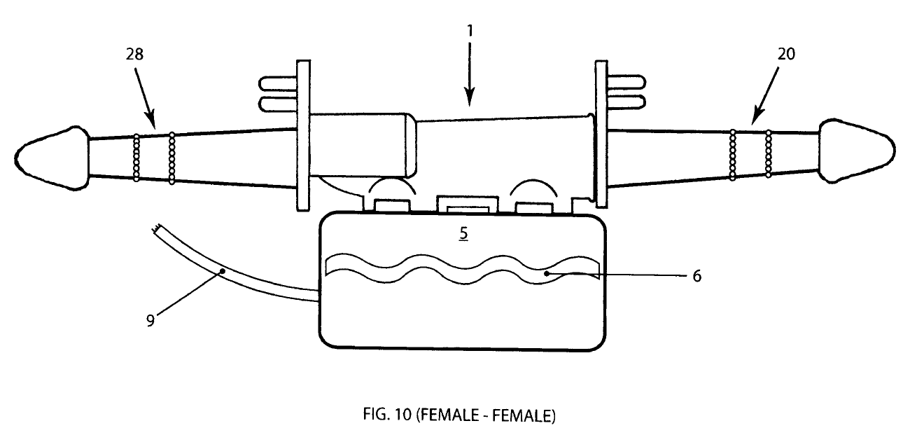

Referring to FIG. 10, in another embodiment of the present invention, a massaging apparatus for a female-female adaptation is shown. In FIG. 10 a unit of female insert 14 and the dildo-1 20 is inserted into the open first end 2 of the vagina 1 and a unit of dildo-2 sleeve 23 and dildo-2 28 is slid over the closed second end of the vagina 1. Due to the configuration of the massaging apparatus, the vagina 1, dildo-1 20 and dildo-2 28 vibrate in a controlled, asynchronous, percussive manner, thereby stimulating the internal and external genitalia of the sex partners. In addition to the vibratory motion, the dildos can be moved horizontally in a stroking motion.

In one embodiment of the present invention dildo-1 20 and dildo-2 28, shown in FIGS. 8 and 10, respectively, may be molded separately, or jointly, to the end(s) of the vagina 1, which may be joined to the vibrator nodes 10 in a same manner as shown in FIG. 4. In this embodiment, the monolithically molded dildos are not detachable and the massaging apparatus is contemplated for use on a female body.

Modifications of the apparatus, procedures and conditions disclosed herein that will still embody the advances and advantages described should suggest themselves to those skilled in the art, and are intended to be encompassed within the spirit of the invention herewith disclosed as well as the scope of the following claims.

What is claimed is:

1. A massaging apparatus comprising:
a vagina in the form of an elongated hollow sheath having a first end and a second end opposite to each other, wherein the first end is open and the second end is closed;
a vibrator coupled to the vagina, wherein the vibrator is at least one of a hand held electric vibrator and a hand held battery powered vibrator; and
a pair of hand grips disposed on both sides of the vibrator.

2. The massaging apparatus of claim 1, wherein the vagina comprises
a pair of mounting cups disposed at bottom of the vagina and coupling the vagina to the vibrator;
a piece of closed cell-foam attached in proximity to the first end of the vagina; and
a vent hole configured at a tip of the second end to remove trapped air formed within the vagina upon inserting a penis into the vagina.

3. The massaging apparatus of claim 1, further comprising
a pair of heat conductors disposed on the vibrator,
a pair of heat strips resting on the heat conductors and disposed within the mounting cups, and
a heat switch disposed on the vibrator, the heat switch capable of activating/deactivating the heat conductors, wherein on activation of the heat conductors, heat is transferred to the vagina through the heat strips.

4. The massaging apparatus of claim 1, wherein the vagina is made up of one of an opaque material and a transparent material.

5. The massaging apparatus of claim 1, wherein the vagina vibrates asynchronously with the vibrator, and the vagina is used by a single male for sexual stimulation.

6. The massaging apparatus of claim 1, further comprising an on-off-speed switch disposed on the vibrator capable of switching on/off the vibrator and adjusting the speed of the vibrator.

7. The massaging apparatus of claim 1, further comprising two dildos removably attached to the first end and the second end of the vagina.

8. The massaging apparatus of claim 7, wherein the vagina and two dildos vibrate asynchronously with the vibrator, and the two dildos are used simultaneously by two females for sexual stimulation.

9. The massaging apparatus of claim 7, wherein the dildos are made of a material selected from the group consisting of polymeric materials, plastic-jelly materials, simulated skin materials, rubber, silicone, and combinations comprising at least one of the foregoing.

10. A massaging apparatus comprising:
a vagina in the form of an elongated hollow sheath having a first end and a second end opposite to each other, wherein the first end is open and the second end is closed;
a vibrator coupled to the vagina, wherein the vibrator is at least one of a hand held electric vibrator and a hand held battery powered vibrator;
a pair of hand grips disposed on both sides of the vibrator; and
a dildo removably attached to the first end of the vagina.

11. The massaging apparatus of claim 10, wherein the vagina comprises a vent hole configured at a tip of the second end to remove trapped air formed within the vagina upon inserting a penis into the vagina.

12. The massaging apparatus of claim 10, wherein the vagina is made up of one of an opaque material and a transparent material.

13. The massaging apparatus of claim 10, wherein the vagina and the dildo vibrate asynchronously with the vibrator, and the dildo is used by a single female for sexual stimulation.

14. The massaging apparatus of claim 10, wherein the dildo is made of a material selected from the group consisting of polymeric materials, plastic-jelly materials, simulated skin materials, rubber, silicone, and combinations comprising at least one of the foregoing.

15. A massaging apparatus comprising:
a vagina in the form of an elongated hollow sheath having a first end and a second end opposite to each other, wherein the first end is open and the second end is closed;
a vibrator coupled to the vagina, wherein the vibrator is at least one of a hand held electric vibrator and a hand held battery powered vibrator;
a pair of hand grips disposed on both sides of the vibrator; and
a dildo removably attached to the second end of the vagina.

16. The massaging apparatus of claim 15, wherein the vagina comprises a vent hole configured at a tip of the second end to remove trapped air formed within the vagina upon inserting a penis into the vagina.

17. The massaging apparatus of claim 15, wherein the vagina is made up of one of an opaque material and a transparent material.

18. The massaging apparatus of claim 15, wherein the vagina and the dildo vibrate asynchronously with the vibrator, and the vagina and the dildo are used simultaneously by a male and a female for sexual stimulation.

19. The massaging apparatus of claim 15, wherein the dildo is made of a material selected from the group consisting of polymeric materials, plastic-jelly materials, simulated skin materials, rubber, silicone, and combinations comprising at least one of the foregoing.
